(19) United States
(12) Patent Application Publication
(10) Pub. No.: US 2019/0008556 A1
(43) Pub. Date: Jan. 10, 2019

(54) INJECTABLE ANCHOR SYSTEM AND METHODS FOR USING THE SAME TO IMPLANT AN IMPLANTABLE DEVICE

(71) Applicant: Micron Devices LLC, Pompano Beach, FL (US)

(72) Inventors: Laura Tyler Perryman, Pompano Beach, FL (US); Chad David Andresen, Miami Beach, FL (US); Benjamin Speck, Miami Beach, FL (US); Graham Patrick Greene, Miami Beach, FL (US)

(21) Appl. No.: 16/030,093

(22) Filed: Jul. 9, 2018

Related U.S. Application Data
(60) Provisional application No. 62/530,501, filed on Jul. 10, 2017.

(51) Int. Cl. A61B 17/34

(52) U.S. Cl. CPC A61B 17/3468; A61B 2017/0023; A61B 17/3415

(57) ABSTRACT

Systems and methods are disclosed for implanting a passive implantable stimulator device to targeted excitable tissue, such as nerves, for treating chronic pain, inflammation, arthritis, sleep apnea, seizures, incontinence, pain associated with cancer, incontinence, problems of movement initiation and control, involuntary movements, vascular insufficiency, heart arrhythmias, obesity, diabetes, craniofacial pain, such as migraines or cluster headaches, and other disorders. In certain embodiments, a device may be used to send electrical energy to targeted nerve tissue by using remote radio frequency (RF) energy without cables or inductive coupling to power a passive implanted wireless stimulator device. The targeted nerves can include, but are not limited to, the spinal cord and surrounding areas, including the dorsal horn, dorsal root ganglion, the exiting nerve roots, nerve ganglions, the dorsal column fibers and the peripheral nerve bundles leaving the dorsal column and brain.

### CROSS-REFERENCE TO RELATED APPLICATION

This application claims the benefit of U.S. Provisional Application No. 62/530,501, filed Jul. 10, 2017, and titled “An Injectable Anchor System and Methods for Using the Same to Implant an Implantable Device,” which is incorporated by reference.

### TECHNICAL FIELD

This application relates generally to systems and methods for implanting implantable stimulators.

### BACKGROUND

Modulation of excitable tissue in the body by electrical stimulation has become an important type of therapy for patients with chronic disabling conditions, including chronic pain, problems of movement initiation and control, involuntary movements, vascular insufficiency, heart arrhythmias and more. A variety of therapeutic intra-body electrical stimulation techniques can treat these conditions. For instance, devices may be used to deliver stimulatory signals to excitable tissue, record vital signs, perform pacing or defibrillation operations, record action potential activity from targeted tissue, control drug release from time-release capsules or drug pump units, or interface with the auditory system to assist with hearing. Typically, such devices utilize a subcutaneous battery operated implantable pulse generator (IPG) to provide power or other charge storage mechanisms.

### BRIEF DESCRIPTION OF THE DRAWINGS

FIG. 1 depicts a high-level diagram of an example of a wireless stimulation system.

FIG. 2 depicts a detailed diagram of an example of the wireless stimulation system.

FIG. 3A shows an example of a loading base.

FIG. 3B shows an example of a loading rod.

FIG. 3C shows an example of a injectroducer assembly.

FIG. 3D shows an example of a deployed anchor.

FIG. 4A to 4G show an example of process of deploying.

Like reference symbols in the various drawings indicate like elements.

### DETAILED DESCRIPTION

In various implementations, systems and methods are disclosed for implanting a passive implantable stimulator device to targeted excitable tissue, such as nerves, for treating chronic pain, inflammation, arthritis, sleep apnea, seizures, incontinence, pain associated with cancer, incontinence, problems of movement initiation and control, involuntary movements, vascular insufficiency, heart arrhythmias, obesity, diabetes, craniofacial pain, such as migraines or cluster headaches, and other disorders. In certain embodiments, a device may be used to send electrical energy to targeted nerve tissue by using remote radio frequency (RF) energy without cables or inductive coupling to power a passive implanted wireless stimulator device. The targeted nerves can include, but are not limited to, the spinal cord and surrounding areas, including the dorsal horn, dorsal root ganglion, the exiting nerve roots, nerve ganglions, the dorsal column fibers and the peripheral nerve bundles leaving the dorsal column and brain, such as the vagus, occipital, trigeminal, hypoglossal, sacral, coccygeal nerves and the like.

A wireless stimulation system can include an implantable stimulator device with one or more electrodes and one or more conductive antennas (for example, dipole or patch antennas), and internal circuitry for frequency waveform and electrical energy rectification. The system may further comprise an external controller and antenna for transmitting radio frequency or microwave energy from an external source to the implantable stimulator device with neither cables nor inductive coupling to provide power.

In various implementations, the wireless implantable stimulator device is powered wirelessly (and therefore does not require a wired connection) and contains the circuitry necessary to receive the pulse instructions from a source external to the body. For example, various embodiments employ internal dipole (or other) antenna configuration(s) to receive RF power through electrical radiative coupling. This allows such devices to produce electrical currents capable of stimulating nerve bundles without a physical connection to an implantable pulse generator (IPG) or use of an inductive coil.

In some implementations, the wireless implantable stimulator device is initially mounted inside the inner lumen of a deployable anchor. At this stage, the wireless implantable stimulator device can be fully pushed inside the inner lumen while the wings of the deployable anchor are pushed to form the anchors. The mounted wireless implantable stimulator device is then placed on the loader and the wings are spread along the two posts on the loader such that the wings are no longer extruding to form the anchors. Thereafter, the injectroducer device may slide along the central groove to load the anchor onto the tip of the needle. The loading may be aided by an air-pressure plunger. Here, the injectducer device may refer to a device that functions as an injector or an introducer. At this position, the wireless implantable simulation device mounted on the deployable anchor can be loaded onto the tip of the needle of the injectroducer. To accomplish this, the plunger handles of the injectducer are pushed to advance the needle into the inner lumen of the deployable anchor. When loaded, the wings may once again be spread back down such that the wings are longer forming the anchors. The injectroducer may then be taken off the loader. At this stage, the injectducer is ready for deploying the anchor device into a subject. In one instance, the needle tip of injectducer may enter a tube (e.g., the lead body enclosing the implanted stimulator device) that has been inserted into the patient through a surgical incision point. The plunger handles of the injectducer may be pushed to advance the anchor assembly down the tube (e.g., lead body). The process may be monitored under X-Ray fluoroscopy. At the implantation site, the wings may be deployed to form the anchors that secure the implantable stimulator device at the implantation site.

Further descriptions of exemplary wireless systems for providing neural stimulation to a patient can be found in commonly-assigned, co-pending published PCT and US applications PCT/US2012/23029 filed Jan. 28, 2011, PCT/US2012/32200 filed Apr. 11, 2011, PCT/US2012/48903, filed Jan. 28, 2011, PCT/US2012/50633, filed Aug. 12, 2011, and PCT/US2012/55746, filed Sep. 15, 2011, US2016/0008602 filed Jul. 19, 2015, the complete disclosures of which are incorporated by reference.

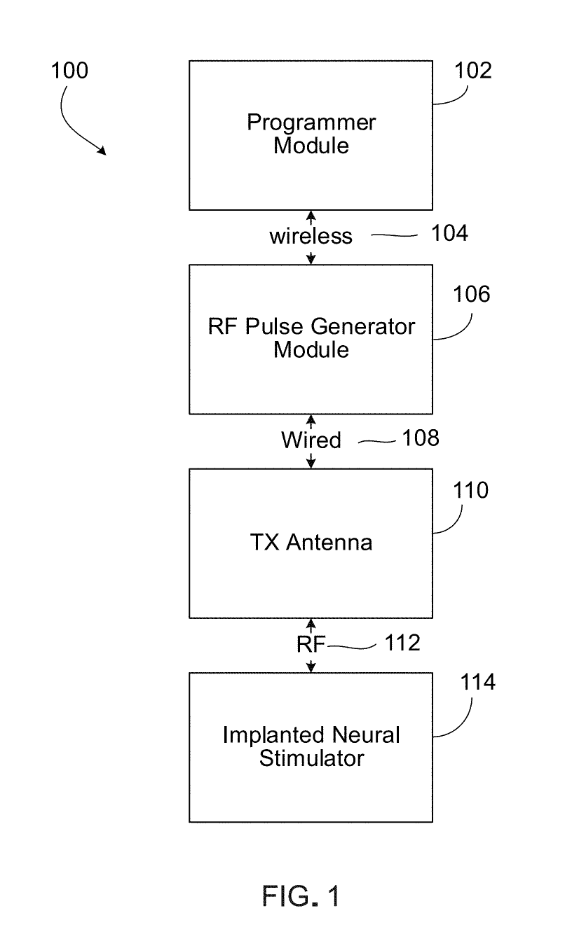

FIG. 1 depicts a high-level diagram of an example of a wireless stimulation system. The wireless stimulation system may include four major components, namely, a programmer module 102, a RF pulse generator module 106, a transmit (TX) antenna 110 (for example, a patch antenna, slot antenna, or a dipole antenna), and an implanted wireless stimulator device 114. The programmer module 102 may be a computer device, such as a smart phone, running a software application that supports a wireless connection 104, such as Bluetooth®. The application can enable the user to view the system status and diagnostics, change various parameters, increase/decrease the desired stimulus amplitude of the electrode pulses, and adjust feedback sensitivity of the RF pulse generator module 106, among other functions.

The RF pulse generator module 106 may include communication electronics that support the wireless connection 104, the stimulation circuitry, and the battery to power the generator electronics. In some implementations, the RF pulse generator module 106 includes the TX antenna embedded into its packaging form factor while, in other implementations, the TX antenna is connected to the RF pulse generator module 106 through a wired connection 108 or a wireless connection (not shown). The TX antenna 110 may be coupled directly to tissue to create an electric field that powers the implanted wireless stimulator device 114. The TX antenna 110 communicates with the implanted wireless stimulator device 114 through an RF interface. For instance, the TX antenna 110 radiates an RF transmission signal that is modulated and encoded by the RF pulse generator module 110. The implanted wireless stimulator device of module 114 contains one or more antennas, such as dipole antenna(s), to receive and transmit through RF interface 112. In particular, the coupling mechanism between antenna 110 and the one or more antennas on the implanted wireless stimulation device of module 114 utilizes electrical radiative coupling and not inductive coupling. In other words, the coupling is through an electric field rather than a magnetic field.

Through this electromagnetic radiative coupling, the TX antenna 110 can provide an input signal to the implanted wireless stimulator device 114. Within the implanted wireless stimulator device 114 are components for demodulating the RF transmission signal, and electrodes to deliver the stimulation to surrounding neuronal tissue. The input signal contains electrical energy to power the creation of a stimulation waveform so that the stimulation waveform can be synthesized and applied at the electrodes. The power level of the electrical energy in the input signal ultimately determines an applied amplitude (for example, power, current, or voltage) of the one or more electrical pulses created using the electrical energy contained in the input signal. In some implementations, the input signal can contain information based on which stimulus waveforms to be synthesized and applied at the electrodes of the implanted wireless stimulator device 114. In one example, the input signal can encode, for example, delay information, or repetition rate information and waveform characteristics as well address information point to a portion of a read-only memory (ROM) on the implantable stimulator device. In this example, the delay information may indicate the amount of latency that the stimulation waveform may be synthesized. Due to the nature of the PDM encoded waveform, an analog waveform can be represented by a stream of single-bit logic values, instead of multi-bit digital code. The address information refers to the storage location on the ROM to retrieve a pulse-density modulated representation of the desired stimulation waveform.

The RF pulse generator module 106 can be implanted subcutaneously, or it can be worn external to the body. When external to the body, the RF generator module 106 can be incorporated into a belt or harness design to allow for electric radiative coupling through the skin and underlying tissue to transfer power and/or control parameters to the implanted wireless stimulator device 114. In either event, receiver circuit(s) internal to the wireless stimulator device 114 can capture the energy radiated by the TX antenna 110 and use this energy to synthesize a stimulation waveform. The receiver circuit(s) may further modify the waveform to create an electrical pulse suitable for the stimulation of neural tissue.

In some implementations, the RF pulse generator module 106 can remotely control the stimulus parameters (that is, the parameters of the electrical pulses applied to the neural tissue) and monitor feedback from the wireless stimulator device 114 based on RF signals received from the implanted wireless stimulator device 114. A feedback detection algorithm implemented by the RF pulse generator module 106 can monitor data sent wirelessly from the implanted wireless stimulator device 114, including information about the energy that the implanted wireless stimulator device 114 is receiving from the RF pulse generator and information about the tissue characteristics the electrode pads see. In order to provide an effective therapy for a given medical condition, the system can be tuned to provide the optimal amount of excitation or inhibition to the nerve fibers by electrical stimulation. A closed loop feedback control method can be used in which the output signals from the implanted wireless stimulator device 114 are monitored and used to determine the appropriate level of neural stimulation current for maintaining effective neuronal activation, or, in some cases, the patient can manually adjust the output signals in an open loop control method.

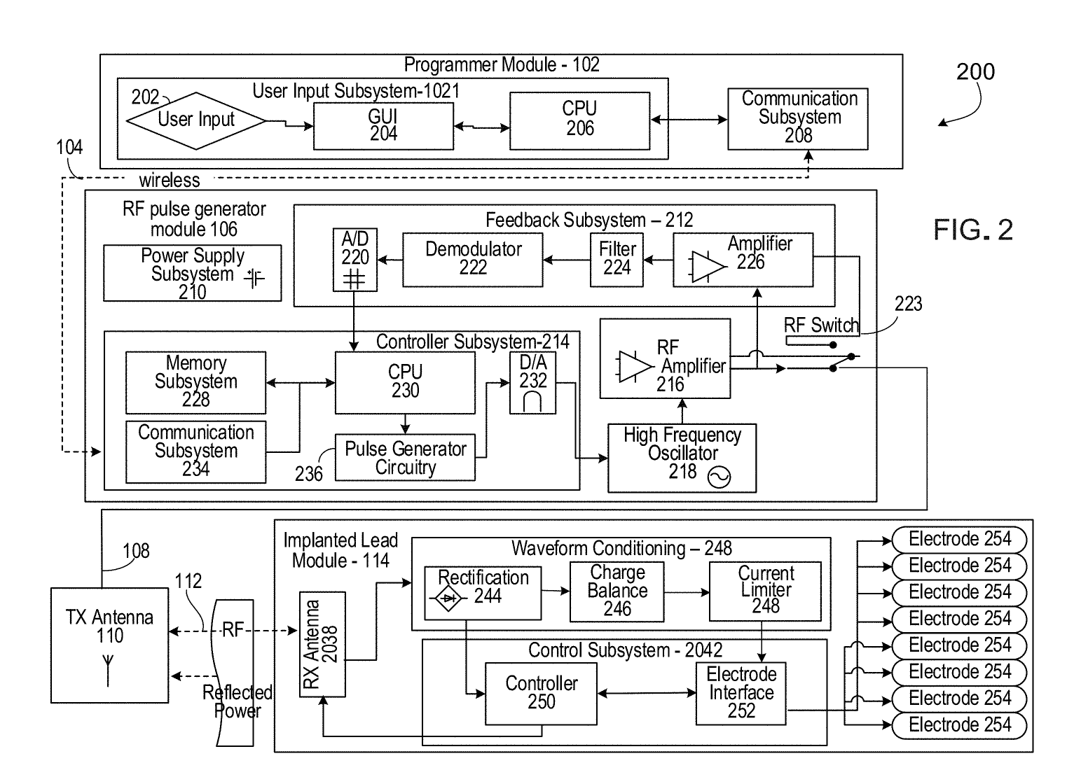

FIG. 2 depicts a detailed diagram of an example of the wireless stimulation system. As depicted, the programming module 102 may comprise user input system 202 and communication subsystem 208. The user input system 221 may allow various parameter settings to be adjusted (in some cases, in an open loop fashion) by the user in the form of instruction sets. The communication subsystem 208 may transmit these instruction sets (and other information) via the wireless connection 104, such as Bluetooth or Wi-Fi, to the RF pulse generator module 106, as well as receive data from module 106.

For instance, the programmer module 102, which can be utilized for multiple users, such as a patient's control unit or clinician's programmer unit, can be used to send information to the RF pulse generator module 106 such that stimulation parameters (e.g., pulse amplitude, pulse frequency, and pulse width) can be controlled. Example ranges of stimulation parameters are shown in Table 1. In this context the term pulse refers to the phase of the waveform that directly produces stimulation of the tissue; the parameters of the charge-balancing phase (described below) can similarly be controlled. The patient and/or the clinician can also optionally control overall duration and pattern of treatment.

TABLE 1
Stimulation Parameter

| | | |
|---|---|---|
| Pulse Amplitude: | 0 to 20 | mA |
| Pulse Frequency: | 0 to 10000 | Hz |
| Pulse Width: | 0 to 2 | ms |

The RF pulse generator module 106 may be initially programmed to meet the specific parameter settings for each individual patient during the initial implantation procedure. Because medical conditions or the body itself can change over time, the ability to re-adjust the parameter settings may be beneficial to ensure ongoing efficacy of the neural modulation therapy.

The programmer module 102 may be functionally a smart device and/or an associated application. The smart device hardware may include a CPU 206 and be used as a vehicle to handle touchscreen input on a graphical user interface (GUI) 204, for processing and storing data.

The RF pulse generator module 106 may be connected via wired connection 108 to an external TX antenna 110. Alternatively, both the antenna and the RF pulse generator are located subcutaneously (not shown).

The signals sent by RF pulse generator module 106 to the implanted wireless stimulator device 114 may include electrical power and configuration data based on which to recover pulse attributes such as stimulus waveform, amplitude, pulse width, and repetition frequency. The configuration data may also include polarity setting information designating the polarity setting for each electrode. The RF pulse generator module 106 can also function as a wireless receiving unit that receives feedback signals from the implanted wireless stimulator device 114. To that end, the RF pulse generator module 106 may contain microelectronics or other circuitry to handle the generation of the signals transmitted to the device 114 as well as handle feedback signals, such as those from the stimulator device 114. For example, the RF pulse generator module 106 may comprise controller subsystem 214, high-frequency oscillator 218, RF amplifier 216, a RF switch, and a feedback subsystem 212.

The controller subsystem 214 may include a CPU 230 to handle data processing, a memory subsystem 228 such as a local memory, communication subsystem 234 to communicate with programmer module 102 (including receiving stimulation parameters from programmer module), pulse generator circuitry 236, and single-bit oversampled (EA) digital/analog (D/A) converters or single-bit controlled full-bridge drivers 232. In other implementations, a Nyquist rate multi-bit D/A converters can also be used for stimulus generation.

The controller subsystem 214 may be used by the patient and/or the clinician to control the stimulation parameter settings (for example, by controlling the parameters of the signal sent from RF pulse generator module 106 to the stimulator device 114). These parameter settings can affect, for example, the power, current level, or shape of the one or more electrical pulses. The programming of the stimulation parameters can be performed using the programming module 102, as described above, to set the repetition rate, pulse width, amplitude, and waveform that will be transmitted by RF energy to the receiving (RX) antenna 238, typically a dipole antenna (although other types may be used), in the implanted wireless stimulation device 214. The clinician may have the option of locking and/or hiding certain settings within the programmer interface, thus limiting the patient's ability to view or adjust certain parameters because adjustment of certain parameters may require detailed medical knowledge of neurophysiology, neuroanatomy, protocols for neural modulation, and safety limits of electrical stimulation.

The controller subsystem 214 may store received parameter settings in the local memory subsystem 228, until the parameter settings are modified by new input data received from the programming module 102. The CPU 206 may use the parameters stored in the local memory to control the pulse generator circuitry 236 to generate a signal that would enable the synthesis of the desired stimulation waveform on the implantable stimulator device 114. The signal can be modulated by a high frequency carrier signal generated by an oscillator 218 in the range from 300 MHz to 8 GHz (preferably between about 700 MHz and 5.8 GHz and more preferably between about 800 MHz and 1.3 GHz). The resulting RF signal may then be amplified by RF amplifier 226 and then sent through an RF switch 223 to the TX antenna 110 to reach through depths of tissue to the RX antenna 238. In the case where a single-bit pulse density modulated waveform is used for stimulus generation, a local oscillator in the range of 1 MHz is used to read-in the bitstream from a ROM device.

In some implementations, the RF signal sent by TX antenna 110 may simply be a power transmission signal used by the wireless stimulation device module 114 to generate electric pulses. In other implementations, a telemetry signal may also be transmitted to the wireless stimulator device 114, which telemetry signal includes instructions about the various operations of the wireless stimulator device 114. The telemetry signal may be sent by the modulation of the carrier signal (through the skin if external, or through other body tissues if the pulse generator module 106 is implanted subcutaneously). The telemetry signal is used to modulate the carrier signal (a high frequency signal) using one of the several modulation methods including On-Off Keying (OOK), Pulse-Amplitude Modulation (PAM), Phase-shift Keying (PSK) and Frequency-Shift Keying (FSK) and does not interfere with the input received on the same stimulator device to power the device. In one embodiment the telemetry signal and powering signal are combined into one signal, where the RF telemetry signal is used to modulate the RF powering signal, and thus the wireless stimulation device is powered directly by the received telemetry signal; separate subsystems in the wireless stimulation device harness the power contained in the signal and interpret the data content of the signal.

The RF switch 223 may be a multipurpose device such as a dual directional coupler, which passes the relatively high amplitude, extremely short duration RF pulse to the TX antenna 110 with minimal insertion loss while simultaneously providing two low-level outputs to feedback subsystem 212; one output delivers a forward power signal to the feedback subsystem 212, where the forward power signal is an attenuated version of the RF pulse sent to the TX antenna 110, and the other output delivers a reverse power signal to a different port of the feedback subsystem 212, where reverse power is an attenuated version of the reflected RF energy from the TX Antenna 110.

During the on-cycle time (when an RF signal is being transmitted to wireless stimulator device 114), the RF switch 223 is set to send the forward power signal to feedback subsystem. During the off-cycle time (when an RF signal is not being transmitted to the wireless stimulator device 114), the RF switch 223 can change to a receiving mode in which the reflected RF energy and/or RF signals from the wireless stimulator device 114 are received to be analyzed in the feedback subsystem 212.

The feedback subsystem 212 of the RF pulse generator module 106 may include reception circuitry to receive and extract telemetry or other feedback signals from the wireless stimulator device 114 and/or reflected RF energy from the signal sent by TX antenna 110. The feedback subsystem may include an amplifier 226, a filter 224, a demodulator 222, and an A/D converter 220.

The feedback subsystem 212 receives the forward power signal and converts this high-frequency AC signal to a DC level that can be sampled and sent to the controller subsystem 214. In this way the characteristics of the generated RF pulse can be compared to a reference signal within the controller subsystem 214. If a disparity (error) exists in any parameter, the controller subsystem 214 can adjust the output to the RF pulse generator 106. The nature of the adjustment can be, for example, proportional to the computed error. The controller subsystem 214 can incorporate additional inputs and limits on its adjustment arrangements such as the signal amplitude of the reverse power and any predetermined maximum or minimum values for various pulse parameters.

The reverse power signal can be used to detect fault conditions in the RF-power delivery system. In an ideal condition, when TX antenna 110 has perfectly matched impedance to the tissue that it contacts, the electromagnetic waves generated from the RF pulse generator 106 pass unimpeded from the TX antenna 110 into the body tissue. However, in real-world applications a large degree of variability may exist in the body types of users, types of clothing worn, and positioning of the antenna 110 relative to the body surface. Since the impedance of the antenna 110 depends on the relative permittivity of the underlying tissue and any intervening materials, and also depends on the overall separation distance of the antenna from the skin, in any given application there can be an impedance mismatch at the interface of the TX antenna 110 with the body surface. When such a mismatch occurs, the electromagnetic waves sent from the RF pulse generator 106 are partially reflected at this interface, and this reflected energy propagates backward through the antenna feed.

The dual directional coupler RF switch 223 may prevent the reflected RF energy propagating back into the amplifier 226, and may attenuate this reflected RF signal and send the attenuated signal as the reverse power signal to the feedback subsystem 212. The feedback subsystem 212 can convert this high-frequency AC signal to a DC level that can be sampled and sent to the controller subsystem 214. The controller subsystem 214 can then calculate the ratio of the amplitude of the reverse power signal to the amplitude of the forward power signal. The ratio of the amplitude of reverse power signal to the amplitude level of forward power may indicate severity of the impedance mismatch.

In order to sense impedance mismatch conditions, the controller subsystem 214 can measure the reflected-power ratio in real time, and according to preset thresholds for this measurement, the controller subsystem 214 can modify the level of RF power generated by the RF pulse generator 106. For example, for a moderate degree of reflected power the course of action can be for the controller subsystem 214 to increase the amplitude of RF power sent to the TX antenna 110, as would be needed to compensate for slightly non-optimum but acceptable TX antenna coupling to the body. For higher ratios of reflected power, the course of action can be to prevent operation of the RF pulse generator 106 and set a fault code to indicate that the TX antenna 110 has little or no coupling with the body. This type of reflected-power fault condition can also be generated by a poor or broken connection to the TX antenna. In either case, it may be desirable to stop RF transmission when the reflected-power ratio is above a defined threshold, because internally reflected power can lead to unwanted heating of internal components, and this fault condition means the system cannot deliver sufficient power to the implanted wireless stimulation device and thus cannot deliver therapy to the user.

The controller 242 of the wireless stimulator device 114 may transmit informational signals, such as a telemetry signal, through the antenna 238 to communicate with the RF pulse generator module 106 during its receive cycle. For example, the telemetry signal may be coupled to the modulated signal on the dipole antenna(s) 238, during the on and off state of the transistor circuit to enable or disable a waveform that produces the corresponding RF bursts necessary to transmit to the external (or remotely implanted) pulse generator module 106. The antenna(s) 238 may be connected to electrodes 254 in contact with tissue to provide a return path. An A/D (not shown) converter can be used to transform stored data to a serialized pattern that can be transmitted on the pulse-modulated telemetry signal from the internal antenna(s) 238 of the wireless stimulator device 114.

A telemetry signal from the implanted wireless stimulator device 114 may include stimulus parameters such as the power or the amplitude of the current that is delivered to the tissue from the electrodes, or impedance of the tissue. The feedback signal can be transmitted to the RF pulse generator module 116 to indicate the strength of the stimulus at the nerve bundle by means of coupling the signal to the implanted RX antenna 238, which radiates the telemetry signal to the external (or remotely implanted) RF pulse generator module 106. The feedback signal can include either or both an analog and digital telemetry pulse modulated carrier signal. Data such as stimulation pulse parameters and measured characteristics of stimulator performance can be stored in an internal memory device within the implanted stimulator device 114, and sent on the telemetry signal. The frequency of the carrier signal may be in the range of at 300 MHz to 8 GHz (preferably between about 700 MHz and 5.8 GHz and more preferably between about 800 MHz and 1.3 GHz).

In the feedback subsystem 212, the telemetry signal can be down modulated using demodulator 222 and digitized by being processed through an analog to digital (A/D) converter 220. The digital telemetry signal may then be routed to a CPU 230 with embedded code, with the option to reprogram, to translate the signal into a corresponding current measurement in the tissue based on the amplitude of the received signal. The CPU 230 of the controller subsystem 214 can compare the reported stimulus parameters to those held in local memory 228 to verify the wireless stimulator device 114 delivered the specified stimuli to tissue. For example, if the wireless stimulation device reports a lower current than was specified, the power level from the RF pulse generator module 106 can be increased so that the implanted wireless stimulator device 114 will have more available power for stimulation. The implanted wireless stimulator device 114 can generate telemetry data in real time, for example, at a rate of 8 Kbits per second. All feedback data received from the implanted stimulator device 114 can be logged against time and sampled to be stored for retrieval to a remote monitoring system accessible by the health care professional for trending and statistical correlations.

The sequence of remotely programmable RF signals received by the internal antenna(s) 238 may be conditioned into waveforms that are controlled within the implantable wireless stimulator device 114 by the control subsystem 242 and routed to the appropriate electrodes 254 that are placed in proximity to the tissue to be stimulated. For instance, the RF signal transmitted from the RF pulse generator module 106 may be received by RX antenna 238 and processed by circuitry, such as waveform generation circuitry 240, within the implanted wireless stimulator device 114 to be converted into electrical pulses applied to the electrodes 254 through electrode interface 252. In some implementations, the implanted wireless stimulator device 114 contains between two to sixteen electrodes 254.

The waveform conditioning and generation circuitry 240 may include a rectifier 244, which rectifies the signal received by the RX antenna 238. The rectified signal may be fed to the controller 242 for receiving encoded instructions from the RF pulse generator module 106. The rectifier signal may also be fed to a charge balance component 246 that is configured to create one or more electrical pulses based such that the one or more electrical pulses result in a substantially zero net charge at the one or more electrodes (that is, the pulses are charge balanced with zero net charge output). The charge-balanced pulses are passed through the current limiter 248 to the electrode interface 252, which applies the pulses to the electrodes 254 as appropriate.

The current limiter 248 insures the current level of the pulses applied to the electrodes 254 is not above a threshold current level. In some implementations, an amplitude (for example, current level, voltage level, or power level) of the received RF pulse (i.e., the input signal) directly determines the amplitude of the stimulus. In this case, it may be particularly beneficial to include current limiter 248 to prevent excessive current or charge being delivered through the electrodes, although current limiter 248 may be used in other implementations where this is not the case. Generally, for a given electrode having several square millimeters surface area, it is the charge per phase that should be limited for safety (where the charge delivered by a stimulus phase is the integral of the current). But, in some cases, the limit can instead be placed on the current, where the maximum current multiplied by the maximum possible pulse duration is less than or equal to the maximum safe charge. More generally, the limiter 248 acts as a charge limiter that limits a characteristic (for example, current or duration) of the electrical pulses so that the charge per phase remains below a threshold level (typically, a safe-charge limit).

In the event the implanted wireless stimulator device 114 receives a “strong” pulse of RF power sufficient to generate a stimulus that would exceed the predetermined safe-charge limit, the current limiter 248 can automatically limit or “clip” the stimulus phase to maintain the total charge of the phase within the safety limit. The current limiter 248 may be a passive current limiting component that cuts the signal to the electrodes 254 once the safe current limit (the threshold current level) is reached. Alternatively, or additionally, the current limiter 248 may communicate with the electrode interface 252 to turn off all electrodes 254 to prevent tissue damaging current levels.

A clipping event may trigger a current limiter feedback control mode. The action of clipping may cause the controller to send a threshold power data signal to the pulse generator 106. The feedback subsystem 212 detects the threshold power signal and demodulates the signal into data that is communicated to the controller subsystem 214. The controller subsystem 214 algorithms may act on this current-limiting condition by specifically reducing the RF power generated by the RF pulse generator, or cutting the power completely. In this way, the pulse generator 106 can reduce the RF power delivered to the body if the implanted wireless stimulator device 114 reports it is receiving excess RF power.

The controller 250 of the stimulator 205 may communicate with the electrode interface 252 to control various aspects of the electrode setup and pulses applied to the electrodes 254. The electrode interface 252 may act as a multiplex and control the polarity and switching of each of the electrodes 254. For instance, in some implementations, the wireless stimulator 106 has multiple electrodes 254 in contact with tissue, and for a given stimulus the RF pulse generator module 106 can arbitrarily assign one or more electrodes to 1) act as a stimulating electrode, 2) act as a return electrode, or 3) be inactive by communication of assignment sent wirelessly with the parameter instructions, which the controller 250 uses to set electrode interface 252 as appropriate. It may be physiologically advantageous to assign, for example, one or two electrodes as stimulating electrodes and to assign all remaining electrodes as return electrodes.

Also, in some implementations, for a given stimulus pulse, the controller 250 may control the electrode interface 252 to divide the current arbitrarily (or according to instructions from pulse generator module 106) among the designated stimulating electrodes. This control over electrode assignment and current control can be advantageous because in practice the electrodes 254 may be spatially distributed along various neural structures, and through strategic selection of the stimulating electrode location and the proportion of current specified for each location, the aggregate current distribution in tissue can be modified to selectively activate specific neural targets. This strategy of current steering can improve the therapeutic effect for the patient.

In another implementation, the time course of stimuli may be arbitrarily manipulated. A given stimulus waveform may be initiated at a time T start and terminated at a time T final, and this time course may be synchronized across all stimulating and return electrodes; further, the frequency of repetition of this stimulus cycle may be synchronous for all the electrodes. However, controller 250, on its own or in response to instructions from pulse generator 106, can control electrode interface 252 to designate one or more subsets of electrodes to deliver stimulus waveforms with non-synchronous start and stop times, and the frequency of repetition of each stimulus cycle can be arbitrarily and independently specified.

For example, a stimulator having eight electrodes may be configured to have a subset of five electrodes, called set A, and a subset of three electrodes, called set B. Set A might be configured to use two of its electrodes as stimulating electrodes, with the remainder being return electrodes. Set B might be configured to have just one stimulating electrode. The controller 250 could then specify that set A deliver a stimulus phase with 3 mA current for a duration of 200 us followed by a 400 us charge-balancing phase. This stimulus cycle could be specified to repeat at a rate of 60 cycles per second. Then, for set B, the controller 250 could specify a stimulus phase with 1 mA current for duration of 500 us followed by a 800 us charge-balancing phase. The repetition rate for the set-B stimulus cycle can be set independently of set A, say for example it could be specified at 25 cycles per second. Or, if the controller 250 was configured to match the repetition rate for set B to that of set A, for such a case the controller 250 can specify the relative start times of the stimulus cycles to be coincident in time or to be arbitrarily offset from one another by some delay interval.

As described above, the wireless stimulator device 114 may include a charge-balancing component 246. Generally, for constant current stimulation pulses, pulses should be charge balanced by having the amount of cathodic current to be equal the amount of anodic current, which is typically called biphasic stimulation. Charge density is the amount of current times the duration it is applied, and is typically expressed in the units uC/cm². In order to avoid the irreversible electrochemical reactions such as pH change, electrode dissolution as well as tissue destruction, no net charge should appear at the electrode-electrolyte interface, and it is generally acceptable to have a charge density less than 30 uC/cm². Biphasic stimulating current pulses ensure that no net charge appears at the electrode after each stimulation cycle and the electrochemical processes are balanced to prevent net dc currents. The wireless stimulator device 114 may be designed to ensure that the resulting stimulus waveform has a net zero charge. Charge balanced stimuli are thought to have minimal damaging effects on tissue by reducing or eliminating electrochemical reaction products created at the electrode-tissue interface.

A stimulus pulse may have a negative-voltage or current, called the cathodic phase of the waveform. Stimulating electrodes may experience both cathodic and anodic phases at different times during the stimulus cycle. An electrode that delivers a negative current with sufficient amplitude to stimulate adjacent neural tissue is called a “stimulating electrode.” During the stimulus phase the stimulating electrode acts as a current sink. One or more additional electrodes act as a current source and these electrodes are called “return electrodes.” Return electrodes are placed elsewhere in the tissue at some distance from the stimulating electrodes. When a typical negative stimulus phase is delivered to tissue at the stimulating electrode, the return electrode has a positive stimulus phase. During the subsequent charge-balancing phase, the polarities of each electrode are reversed.

In some applications, the charge balance component 246 can include a blocking capacitor(s) placed electrically in series with the stimulating electrodes and body tissue, between the point of stimulus generation within the stimulator circuitry and the point of stimulus delivery to tissue. In these implementations, the tissue impedance and additional filtering capacitance can form an AC band-pass filter that reconstructs the charge balanced waveform. In a multi-electrode stimulator, one charge-balance capacitor(s) may be used for each electrode or a centralized capacitor(s) may be used within the stimulator circuitry prior to the point of electrode selection. The AC high-pass filter can block direct current (DC). However, in some instances, it can also prevent low-frequency alternating current (AC) from passing to the tissue. The frequency below which the series RC network essentially blocks signals is commonly referred to as the cutoff frequency, and in one embodiment the design of the stimulator system may ensure the cutoff frequency is low enough such that the desired stimulus waveform can pass without a significant filtering. In this embodiment as disclosed herein, the wireless stimulator may have a charge-balance capacitor with a value chosen according to the measured series resistance of the electrodes and the tissue environment in which the stimulator is implanted. By selecting a specific capacitance value, the cutoff frequency of the AC high-pass filter in this embodiment can be, for example, at or below the fundamental frequency of the stimulus pulse.

In other implementations, the cutoff frequency may be chosen to be at or above the fundamental frequency of the stimulus, and in this scenario the stimulus waveform created prior to the charge-balance capacitor, called the drive waveform, may be designed to be non-stationary, where the envelope of the drive waveform is varied during the duration of the drive pulse. For example, in one embodiment, the initial amplitude of the drive waveform is set at an initial amplitude Vi, and the amplitude is increased during the duration of the pulse until it reaches a final value k*Vi. By changing the amplitude of the drive waveform over time, the shape of the stimulus waveform passed through the charge-balance capacitor is also modified. The shape of the stimulus waveform may be modified in this fashion to create a physiologically advantageous stimulus.

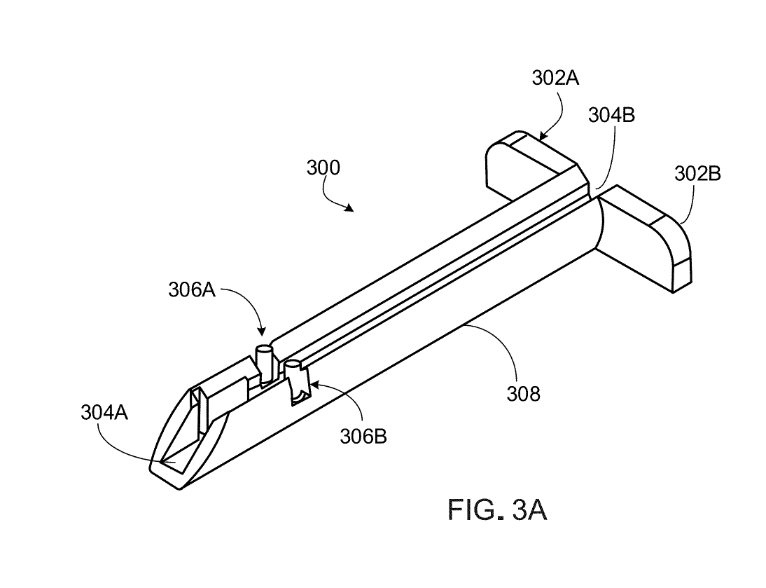

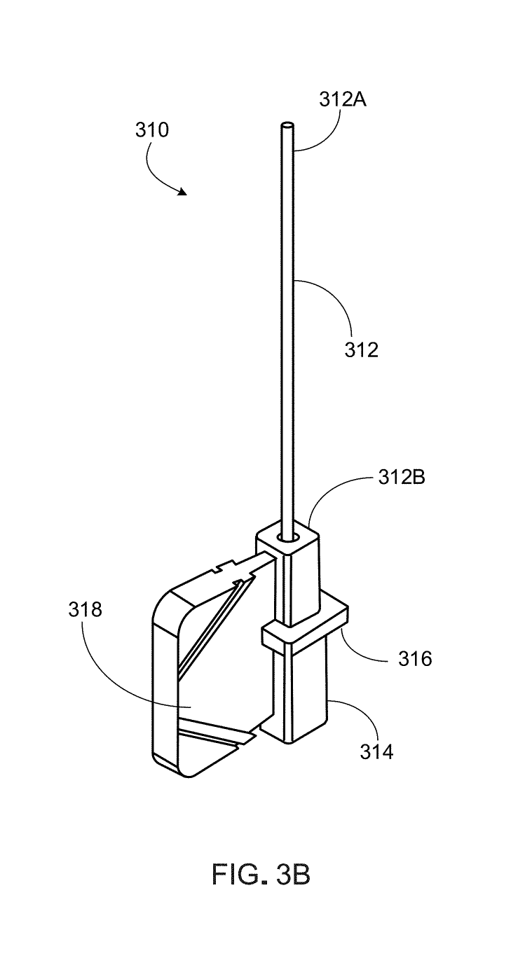

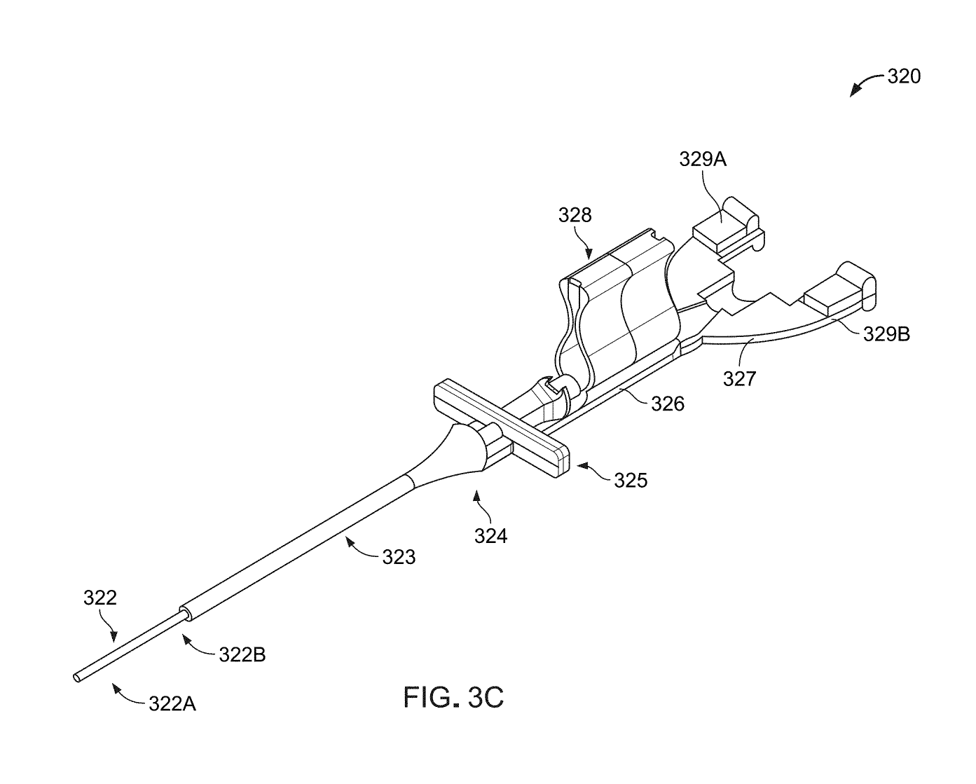

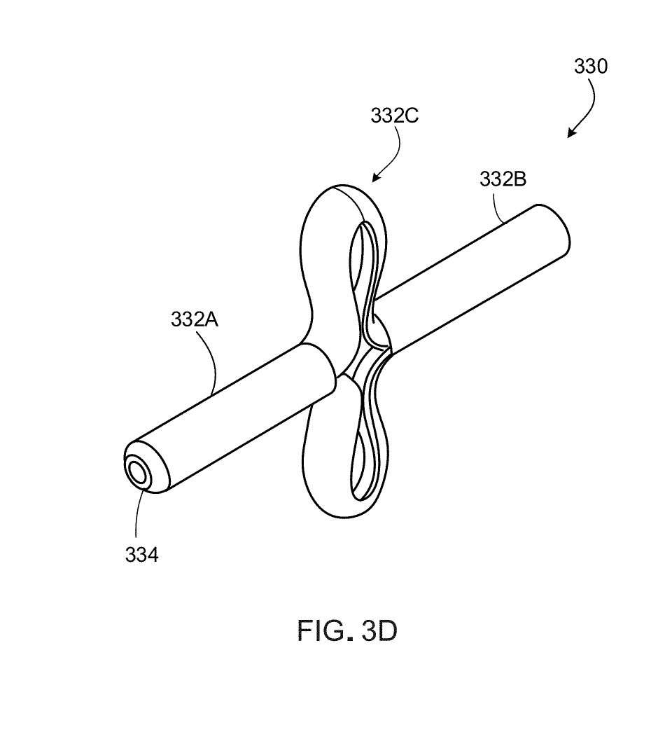

FIG. 3A shows an example of a loading base 300. The loading base 300 includes an end bar that has a left side portion 302A and a right side portion 302B. The loading base 300 also includes a central groove having a distal end 304A and a proximal end 304B that is located between left side portion 302A and right side portion 302B. Towards the distal end, two posts 306A and 306B are distributed along a notch. The loading base 300 may be used to load stimulator and anchor assembly onto the injectroducer.

FIG. 3B shows an example of a loading rod 310. The loading rod 310 includes a pin 312 that has a proximal end 312B and a distal end 312A. The pin may taper to a larger diameter at the proximal end 312B. The pin 312 joins a base portion 314 at the proximal end 312B. The base portion 314 includes a handle 318 and a knob 316.

FIG. 3C shows an example of a injectroducer assembly 320. The assembly 320 includes a needle tip 322 that includes a distal end 322A and a proximal end 322B. The needle tip 322 is slidable into a needle body at the proximal end 322B. The needle body 323 joins plunger handle 325 at needle base 324. Clip 328 snatches handle area 326 that is proximal to plunger handle 325. Tail area includes base 327 and end portions 329A and 329B.

FIG. 3D shows an example of a deployed anchor 330, which can be an extruded tube with slits. Anchor 330 has a core 334 that includes a central lumen. The central lumen can enclose a stimulator device or catheter device. Anchor 330 has a first end jacket 332A and a second end jacket 332B on both sides. Anchor 330 has a center jacket 332C that is slidable over the core to form the anchor, as deployed in the illustration.

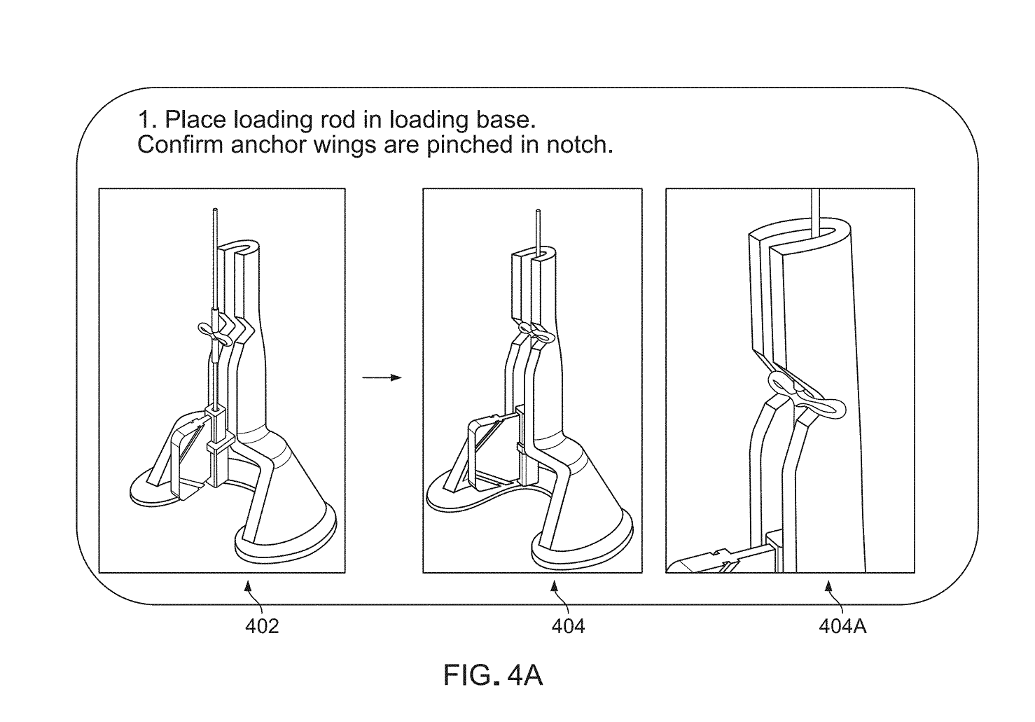

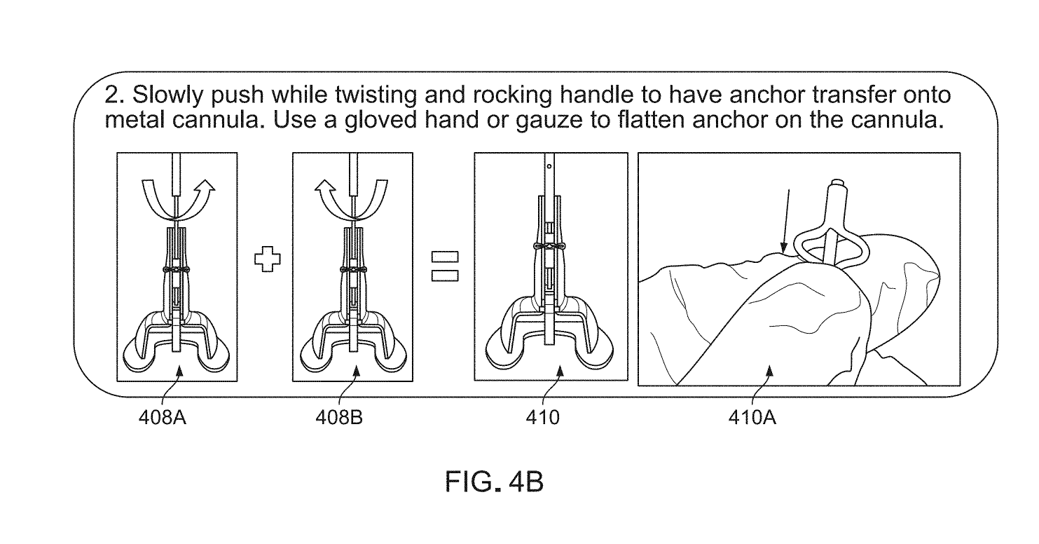

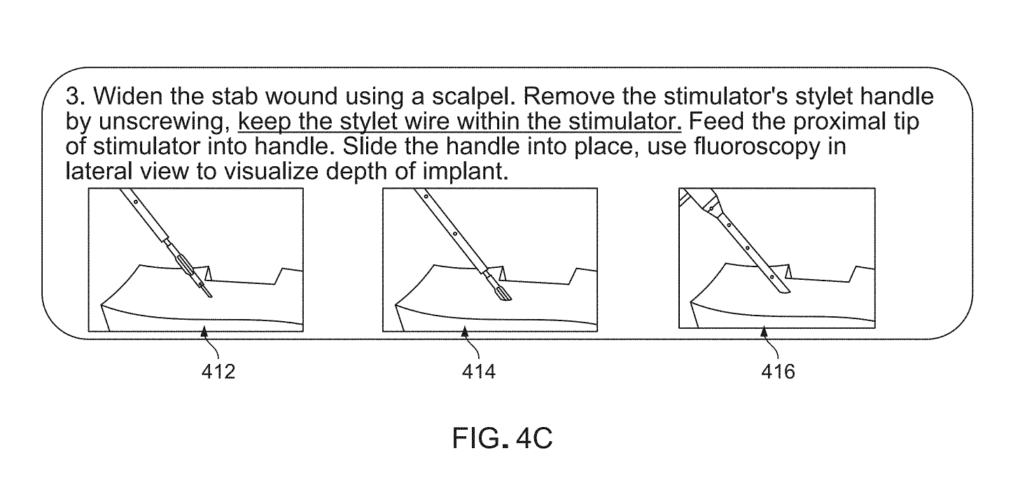

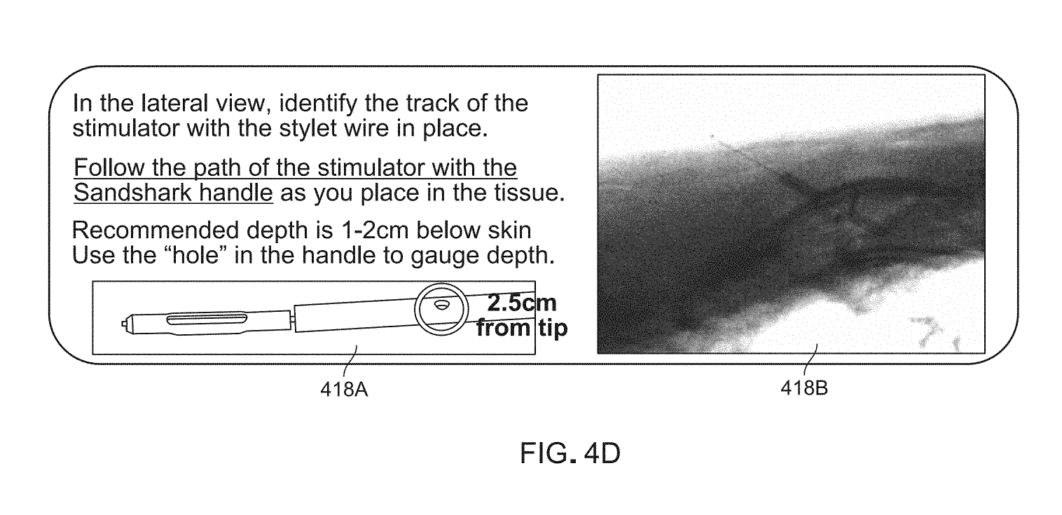

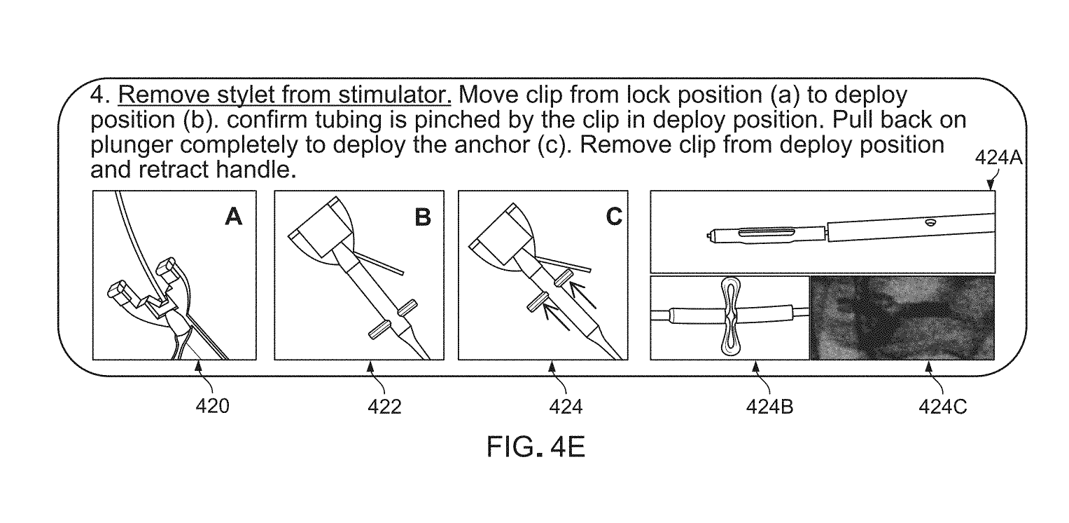

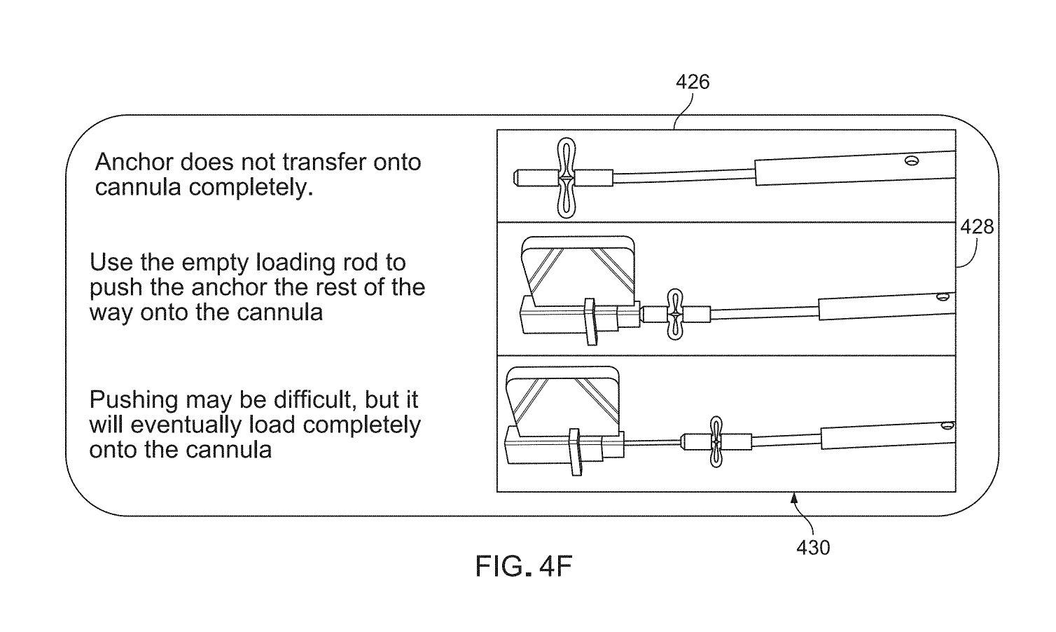

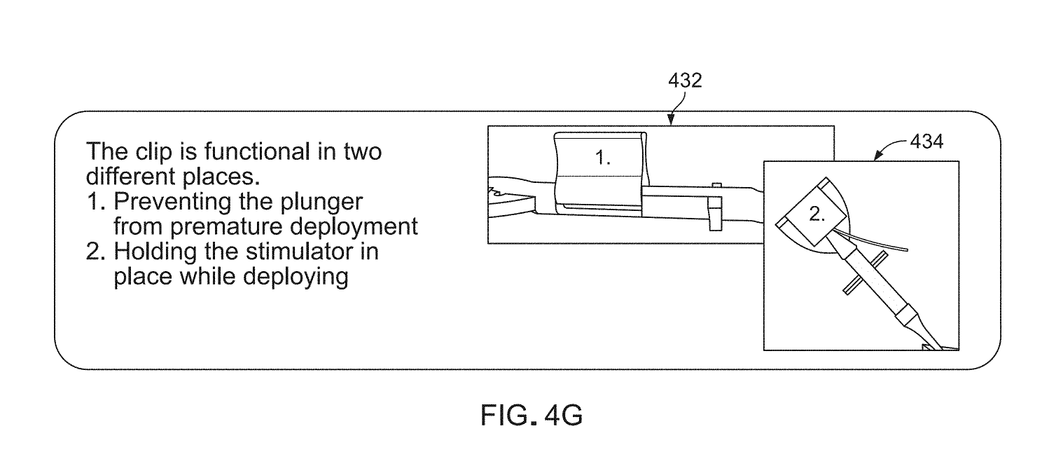

An example of a process of deploying the anchor to secure an implantable stimulator device is shown in FIGS. 4A to 4G. Anchor 330 may be deployed on the central pin 312A of loading rod 310 (402). In this position, anchor 330 is deployed to form the anchor wings. Anchor 330 mounted on loading ping 310 will then be placed in the central groove of loading base 300 (404). The anchor wings of the deployed anchor 330 may fit into the notch at the distal end so that the lateral sides of the anchor wings are pinched in the notch at the two posts 306A and 306B, as illustrated in the amplified view of 404A.

Loading rod 310 may then be slowly pushed with handle 318 gently twisted and rocked, as illustrated in 408A and 408B, to have anchor 330 transferred onto metal cannula on the distal end 322A of injectroducer assembly 320.

Referring to FIGS. 4A to 4G, the wireless implantable stimulator device is initially mounted inside the inner lumen of a deployable anchor. At this stage, the wireless implantable stimulator device can be fully pushed inside the inner lumen while the wings of the deployable anchor are pushed to form the anchors, as illustrated in view 410 and amplified in view 410A. Here, the anchor may not transfer onto the cannula completely. The empty loading rod may be used to push the anchor the rest of the way onto the cannula, as illustrated in views 426, 428, and 430. In some cases, pushing may experience some resistance. The mounted wireless implantable stimulator device is then placed on the loader and the wings are spread along the two posts on the loader such that the wings are no longer extruding to form the anchors. Thereafter, the injectroducer device may be pushed to load the mounted stimulator device onto the tip of the metal cannula. The loading may be by an air-pressure plunger. Here, the injectducer device may refer to a device that functions as an injector or an introducer. At this position, the wireless implantable simulation device mounted on the deployable anchor can be loaded onto the tip of the cannula of the injectroducer. To accomplish this, the handles of the injectducer are pushed to advance the tip of the cannula into the inner lumen of the deployable anchor. When loaded, the wings may once again be spread back down such that the wings are longer forming the anchors. The injectroducer may then be taken off the loader. At this stage, the injectducer is ready for deploying the wireless stimulator device into a subject. In one instance, the cannula tip of injectducer may enter a tube (e.g., the lead body enclosing the implanted stimulator device) that has been inserted into the patient through a surgical incision point. View 424A illustrate the relative positions of the assembly and the lead body. The handles of the injectducer may be pushed to advance the stimulator and anchor assembly down the tube (e.g., lead body), as illustrative in views 412, 414, and 416, to deliver the assembly into the patient's body. In this implantation process, the stylet may be removed from the stimulator. The clip may be removed from the locking position corresponding to views 420 and 432 to the deploy position shown in views 422, 424, and 434. Then handles of the injectducer may then be pushed, as illustrated in views 424. The process may be monitored under X-Ray fluoroscopy, as illustrated in views 418B and 424C. At the implantation site, the wings may be deployed to form the anchors that secure the implantable stimulator device at the implantation site, as shown in view 424B. The implantation location can be a few centimeters (e.g., 1-3 cm) below the skin, as illustrated in 418A.

A number of implementations have been described. Nevertheless, it will be understood that various modifications may be made. Accordingly, other implementations are within the scope of the following claims.

What is claimed is:

1. An implantable anchor for anchoring an implantable stimulator device or catheter device within a subject, the anchor comprising:
an anchor body that includes a lumen extending from a first end of the anchor body to a second end of the anchor body, the lumen is sized and shaped to enclose the passive implantable stimulator device.

2. The implantable anchor of claim 1, wherein the implantable anchor is configurable to be shaped into two configurations including:
a first configuration corresponding to a deployed state in which the lumen becomes restricted in inner diameter size at a first location such that the lumen physically grips to the passive implantable stimulator enclosed therein while extruding perpendicularly to a longitudinal axis of the body at a second location where the lumen is not restricted and a pair of wings are formed; and
a second form corresponding to a non-deployed state in which the lumen maintains inner diameter size and without extruding perpendicularly to the longitudinal axis of the body.

3. The implantable anchor of claim 1, wherein the lumen is sized and shaped to accommodate a deployable handle and an injectroducer device therein that extends through the lumen and barely extrudes outside the lumen openings.

4. The implantable anchor of claim 1, wherein the anchor is made of a flexible polymer material with or without radio-opaque properties.

5. The implantable anchor of claim 1, wherein the anchor has a tapered, beveled, or angled tip to on the leading edge to reduce friction and hang-up during implantation.

6. The implantable anchor of claim 1, wherein the anchor is configured to rest on a loading rod having a diameter smaller than the anchor, wherein the anchor is configured to be loaded onto a deployment handle on the loading rod.

7. The implantable anchor of claim 1, comprising one or more accessories suitable for loading the anchor onto a deployable handle immediately before the anchor is to be implanted, the one or more accessories including the deployable handle, a loading tool, and a loading rod.

8. The implantable anchor of claim 6, wherein the loading handle integrally connects to a movable cannula that is extendable at two or more positions to deploy the anchor.

9. The implantable anchor of claim 6, wherein the cannula comprises lubricious material to render the surface lubricious.

10. The implantable anchor of claim 6, wherein the cannula has a tapered, beveled, or angled tip for loading the anchor.

11. The implantable anchor of claim 6, wherein the loading tool is configurable to mechanically push, stretch, or dilate the anchor to allow the cannula accessory to pass through the lumen of the anchor.

12. The implantable anchor of claim 6, wherein the loading tool is sized and shaped to be operable by hand to ease a loading force to push the anchor from the loading rod to the cannula.

13. The implantable anchor of claim 6, wherein the loading rod is tapered in diameter.

14. The implantable anchor of claim 6, wherein the loading rod has a recessed cavity that allows the cannula tip to enter but does not allow the anchor to enter.

15. The implantable anchor of claim 6, wherein the implantable anchor is disposable for single use only and is a part of a sterile or non-sterile anchoring system for neurostimulators, catheters, cannula, or leads.

16. The implantable anchor of claim 6, wherein a locking mechanism is incorporated into the deployable handle which locks the stimulator in place during deployment.

17. The implantable anchor of claim 6, wherein a removable connects to the deployable handle which locks the stimulator in place during deployment.

18. The implantable anchor of claim 6, wherein a locking mechanism is incorporated into the deployable handle which prevents premature deployment of the anchor.

19. The implantable anchor of claim 6, wherein a removable lock mechanism connects to the deployable handle which prevents premature deployment of the anchor.

20. The implantable anchor of claim 6, wherein the deployable handle includes tactile feedback or sound mechanism that alerts the user to change in sliding position.

21. The implantable anchor of claim 6, wherein the deployable handle has built-in features or printed graphics which identify orientation of the bevel tip direction.
